SEQUENCE LISTING (4 sequences)

SEQ ID NO: 1 - DNA, 20 bp, synthetic construct
  source 1..20: mol_type = other DNA
cagctcagctcagtacctca

SEQ ID NO: 2 - DNA, 20 bp, synthetic construct
  source 1..20: mol_type = other DNA
cttgaggccctgattaccca

SEQ ID NO: 3 - DNA, 23 bp, synthetic construct
  source 1..23: mol_type = other DNA
gttgtaattgagaaagacaattg

SEQ ID NO: 4 - DNA, 23 bp, synthetic construct
  source 1..23: mol_type = other DNA
tacagttaagataacttcgacag

(19) United States
(12) Patent Application Publication
(10) Pub. No.: US 2026/0207457 A1
(43) Pub. Date: Jul. 23, 2026

(54) COMPOSITION FOR CARING FOR KERATIN MATERIALS, COMPRISING AT LEAST INDOLE-3-LACTIC ACID, INDOLE-3-CARBOXALDEHYDE, INDOLE-3-ACETIC ACID AND AN ADJUVANT, USES AND PROCESS IMPLEMENTING THE COMPOSITION

(71) Applicant: L'OREAL, Paris (FR)

(72) Inventors: Pascal HILARIE, Tours Cedex 2 (FR); Leslie LANDEMAINE, Aulnay-Sous-Bois (FR); Cecile CLAVAUD, Aulnay-Sous-Bois (FR); Mathias RICHARD, Jouy En Josas (FR)

(21) Appl. No.: 19/141,741

(22) PCT Filed: Dec. 20, 2023
(86) PCT No.: PCT/EP2023/087036

§ 371 Date: Jun. 20, 2025

(30) Foreign Application Priority Data
Dec. 21, 2022 (FR) FR2214153

(51) Int. Cl. A61K 8/49; A61K 8/99; A61K 31/4045; A61K 31/405; A61Q 19/00

(52) U.S. Cl. CPC A61K 8/492; A61K 8/99; A61K 31/4045; A61K 31/405; A61Q 19/007

(57) ABSTRACT

A composition including, in a physiologically acceptable medium: at least indole-3-lactic acid; indole-3-carboxaldehyde; indole-3-acetica acid; and/or one of their salts; and at least one adjuvant. Also the use of such a composition for preventing a reduction in and/or reinforcing the skin barrier function in an individual, for improving skin moisturization, for improving the quality of the skin, in particular of dry skin, for preventing and/or treating cosmetic manifestations associated with dry skin, in particular selected from tautness, itching, feelings of discomfort, a dull complexion, skin roughness and a marked microrelief of the skin, and/or for preventing and/or treating pruritus, in particular pruritus of skin affected by a dermatological disorder such as atopic dermatitis and/or for preventing and/or treating atopic dermatitis. Also a non-therapeutic cosmetic process for caring for keratin materials, including at least one step of topical application to said keratin materials of such a composition.

### TECHNICAL FIELD

The present invention relates to the use of a mixture of indoles for improving the barrier function and moisturizing the skin.

More particularly, the present invention aims to propose a composition, particularly a cosmetic composition, in particular for caring for keratin materials, comprising, in a physiologically acceptable medium:
at least indole-3-lactic acid and/or a salt thereof, indole-3-carboxaldehyde and/or a salt thereof, and indole-3-acetic acid and/or a salt thereof; and
at least one adjuvant selected from the group constituted by fatty substances, organic solvents selected from lower monoalcohols having from 1 to 5 carbon atoms such as ethanol, isopropanol, tert-butanol; polyols such as propylene glycol, pentylene glycol, caprylyl glycol, hexylene glycol (or 2-methyl-2,4-pentanediol) and polyethylene glycols; polyol ethers such as dipropylene glycol monomethyl ether, ionic or nonionic, hydrophilic or lipophilic, thickeners; softeners; opacifiers; stabilizers; silicones; antifoams; fragrances; preservatives; anionic, cationic, nonionic, zwitterionic or amphoteric surfactants; fillers; polymers; propellants; and mixtures thereof.

The invention also relates to the cosmetic use, particularly topical cosmetic use, of such a composition for preventing a reduction in and/or reinforcing the skin barrier function in an individual.

Moreover, it relates to the cosmetic use, particularly topical cosmetic use, of such a composition for improving skin moisturization.

Furthermore, it relates to the cosmetic use, particularly topical cosmetic use, of such a composition for improving the quality of the skin, in particular of dry skin.

Furthermore, it relates to the cosmetic use, particularly topical cosmetic use, of such a composition for preventing and/or treating cosmetic manifestations associated with dry skin, in particular selected from tautness, itching, feelings of discomfort, a dull complexion, skin roughness and a marked microrelief of the skin.

Moreover, it relates to the cosmetic use, particularly topical cosmetic use, of such a composition for preventing and/or treating pruritus, in particular pruritus of skin affected by a dermatological disorder such as atopic dermatitis.

It also relates to the cosmetic use, particularly topical cosmetic use, of such a composition for preventing and/or treating atopic dermatitis.

Finally, the invention relates to a non-therapeutic cosmetic process for caring for keratin materials, in particular the skin, particularly dry skin, comprising the topical application to these keratin materials of such a composition.

### PRIOR ART

The skin is a tissue, the cells of which are joined together and integrally attached to each other. The skin tissue forms an outer covering comprising sebaceous or sweat glands, and hair follicles. The skin is an epithelium which undergoes continual renewal. Renewal, or desquamation, is a coordinated and finely regulated process resulting in the imperceptible and invisible removal of the surface cells.

Human skin is constituted of two compartments, namely an upper compartment, the epidermis, and a deep compartment, the dermis.

The epidermis is conventionally divided into a basal layer of keratinocytes that constitutes the germinal layer of the epidermis, a spinous layer constituted of several layers of polyhedral cells positioned on the germinal layers, one to three “granular” layers constituted of flattened cells containing distinct cytoplasmic inclusions, keratohyalin granules, and finally a set of upper layers referred to as the cornified layers (or stratum corneum), constituted of keratinocytes at the terminal stage in their differentiation, referred to as corneocytes. The organization, and above all the cohesion, between these various cell layers is made possible by a set of intercellular junctions including adherens junctions which contribute to maintaining homeostasis in the epithelial tissue. These adherens junctions are particularly referred to as desmosomes. Desmosomes are composed of transmembrane proteins from the cadherin family, such as desmoglein. These junctions have an essential role in the structure, maintenance and architectural cohesion of the epidermis and enable transmission and damping of mechanical forces acting on the keratinocytes. These adherens junctions are predominantly found in the intermediate layers of the epidermis up to the uppermost layers, namely the cornified layer composed of corneocytes.

Corneocytes are a nuclear cells mainly constituted of a fibrous material containing cytokeratins, surrounded by a cornified envelope, constituted particularly of the protein filaggrin. Filaggrin aggregates the keratin filaments into macrofibrils in the lower part of the cornified layer. When it comes close to the skin's surface, filaggrin is mainly broken down into free amino acids which form a major portion of a highly hygroscopic complex, natural moisturizing factor (NMF). NMF is important for maintaining the moisturization of the cornified layer and the suppleness of the skin.

New keratinocytes are constantly being produced to compensate for the continuous loss of epidermal cells at the cornified layer by a mechanism referred to as desquamation.

Nevertheless, an imbalance between the production of cells at the basal layer and the rate of desquamation may particularly lead to the formation of scales on the surface of the skin.

Similarly, a lack of the terminal differentiation of the cells of the stratum corneum, for various reasons, can result in the formation of large, thick cell clusters which are visible to the naked eye and called “squamae”, or in other situations, in a thinning of the stratum corneum.

In addition, a reduction in the whole of the junction system which is responsible for cell-cell cohesion directly results in a reduction in the efficacy of the skin's barrier function.

Thus, one or more of these internal factors may lead to a weakness in the barrier properties of the epidermis, to chronic dehydration of the stratum corneum, to a loss of mechanical elasticity, to tautness, and also to a loss of radiance and transparency of the skin.

In parallel, weakening of the skin barrier can also occur in the presence of external attacks, particularly chosen from irritants (detergents, acids, bases, oxidizers, reducing agents, concentrated solvents, toxic gas or fumes), thermal or climatic imbalances (cold, drought, radiation), xenobiotics (undesirable microorganisms, allergens, pollutants).

One of the critical steps in the process of terminal differentiation of the stratum corneum is the crosslinking of the precursor proteins of the cornified envelope (CE). This phenomenon has an essential role in the development and maintenance of skin cohesion, of the physical properties of the skin such as the barrier function, and is a crucial step in the process of terminal differentiation. The cornified envelope is an essential component of corneocytes.

The moisturizing active ingredients conventionally used, such as humectants, moisturising polymers or occlusive fatty substances such as liquid petroleum jelly, temporarily modify the surface properties of the skin. These active ingredients may lead to mechanical softening of the stratum corneum, to an increase in the state of moisturization thereof and/or an improvement in the skin's microrelief by the formation of a film at the surface of the skin. However, these effects are not necessarily particularly persistent over time and disappear after cleansing the skin. Moreover, these active ingredients can be eliminated by cleansing actions.

In order to overcome these disadvantages, it may be advantageous to opt for active ingredients that have a beneficial effect on biological pathways and biomarkers involved in the skin barrier function.

Aside from the key factors involved in the skin barrier function and mentioned above, which are, particularly, filaggrin and adherens junctions (desmoglein), the tryptophan metabolic pathway, namely the aryl hydrocarbon receptor (AHR) pathway, is of particular interest. This is because this pathway is known to be involved in various skin disorders such as accelerated ageing and inflammation (Parrado, C. et al. Front. Pharmacol. 10, (2019); Krutmann, J. Dermatol. Sci. 85, 152-161 (2017); Vogeley, C., Int. J. Mol. Sci. 20, (2019).—Hidaka, T., Front. Med. 6, (2019); Stockinger, B Annu. Rev. Immunol. 32, 403-432 (2014)).

It is also known to be essential for the integrity of the skin barrier (Haas, K. et al. J. Invest. Dermatol. 136, 2260-2269 (2016)) and the activation thereof, particularly by the genes CYP1A1 and/or OVOL1, makes it possible to improve the skin barrier in the context of atopic dermatitis (Furue, M., Hashimoto-Hachiya, A. & Tsuji, G. Int. J. Mol. Sci. 20, (2019)).

Thus, there is a need to identify new active ingredients which make it possible to stimulate not only the tryptophan metabolic pathway, namely the aryl hydrocarbon receptor (AHR) pathway, but also to stimulate expression of filaggrin and/or of adherens junctions such as desmoglein.

There is a need for active ingredients that enable the skin, in particular dry skin, to maintain its barrier function.

There is a need for active ingredients that make it possible to prevent a reduction in, and/or to reinforce, the skin barrier function in an individual.

There is also a need for active ingredients that make it possible to improve skin moisturization.

There is additionally a need for active ingredients that make it possible to improve the quality of the skin, in particular of dry skin.

There is additionally a need for active ingredients that make it possible to prevent and/or treat cosmetic manifestations associated with dry skin, in particular selected from tautness, itching, feelings of discomfort, a dull complexion, skin roughness and a marked microrelief of the skin.

There is also a need for active ingredients that make it possible to prevent and/or treat pruritus, in particular pruritus of skin affected by a dermatological disorder such as atopic dermatitis.

There is additionally a need for active ingredients that make it possible to prevent and/or treat atopic dermatitis.

### DISCLOSURE OF THE INVENTION

The aim of the present invention is to solve the abovementioned technical problems.

Indeed, the inventors have now discovered that a mixture of indoles according to the present invention makes it possible to overexpress genes for activating the AHR pathway (particularly the genes CYP1A1 and/or OVOL1 described by Furue et al. Int. J. Mol. Sci. 20, (2019)) and genes for stimulating epidermal differentiation (FLG—Filaggrin and/or DSG1—Desmoglein 1), and thereby to prevent a reduction in, and/or to reinforce, the skin barrier function in an individual. This mixture is therefore advantageous in improving the barrier function and moisturization of the skin.

### SUMMARY OF THE INVENTION

Thus, according to a first aspect, the present invention relates to a composition, particularly a cosmetic composition, in particular for caring for keratin materials, comprising, in a physiologically acceptable medium:
at least indole-3-lactic acid and/or a salt thereof, indole-3-carboxaldehyde and/or a salt thereof, and indole-3-acetic acid and/or a salt thereof; and
at least one adjuvant selected from the group constituted by fatty substances, organic solvents selected from lower monoalcohols having from 1 to 5 carbon atoms such as ethanol, isopropanol, tert-butanol; polyols such as propylene glycol, pentylene glycol, caprylyl glycol, hexylene glycol (or 2-methyl-2,4-pentanediol) and polyethylene glycols;
polyol ethers such as dipropylene glycol monomethyl ether, ionic or nonionic, hydrophilic or lipophilic, thickeners; softeners; opacifiers; stabilizers; silicones; antifoams; fragrances;
preservatives; anionic, cationic, nonionic, zwitterionic or amphoteric surfactants; fillers;
polymers; propellants; and mixtures thereof.

A “polyol” that is suitable for use in the invention is intended to be a compound of linear, branched or cyclic, saturated or unsaturated alkyl type, bearing on the alkyl chain at least two —OH functions, in particular at least three —OH functions and more particularly at least four —OH functions.

The polyols that are suitable for formulating a composition according to the present invention are in particular those containing particularly from 2 to 32 carbon atoms, preferably from 3 to 16 carbon atoms.

By “filler”, it must be understood colorless or white solid particles of all shapes, which are in an insoluble form and dispersed in the medium of the composition. Mineral or organic in nature, they give body or rigidity to the composition and/or softness.

The fillers used in the compositions according to the present invention can be of lamellar, globular, spherical, fiber shapes or any other intermediate shape between these defined shapes.

The fillers in the context of the invention may or may not be superficially coated, and, in particular, they may be surface treated with silicones, amino acids, fluorinated derivatives or any other substance promoting dispersion and compatibility of filler in the composition.

The filler can be inorganic or organic.

The mineral fillers can be chosen from synthetic or natural mica; silica powder; talc; kaolin; boron nitride or mixtures thereof.

The organic fillers can be chosen from:
organopolysiloxane powders coated with silicone resin, such as those sold under the trade names “KSP-100”, “KSP-101”, “KSP-102”, “KSP-103”, KSP-104”, “KSP-105” by Shin Etsu,
polymethylsilsesquioxane powders, such as those sold under the trade name TOSPEARL by Momentive Performance Materials;
polyamide powders also known as Nylon such as Nylon-1 (Polyamide 1), Nylon-12 (Polyamide 12), such as those sold under the trade names ORGASOL by Arkema Nylon-66 (Polyamide 66); Nylon-6 (Polyamide 6);
polyethylene powders;
microspheres based on acrylic copolymers, such as those made of ethylene glycol dimethacrylate/lauryl methacrylate copolymer sold by Dow Corning under the trade name POLYTRAP;
expanded powders such as hollow microspheres and in particular, microspheres sold under the trade name EXPANCEL by Nouryon;
methyl polymethacrylarte microspheres, such as those sold under the trade name MICROSPHERE M-100 by Matsumoto or under the trade name COVABEAD LH85 by Sensient;
ethylene-acrylate copolymer powders, such as those sold under the trade name FLOBEADS by Sumitomo Seika Chemicals;
natural organic material powders such as starch powders particularly corn, wheat or rice starches, optionally crosslinked, such as starch powders crosslinked with octenylsuccinate anhydride, such as those marketed under the trade name DRY-FLO by Nouryon;
poly-p-phenylene terephtamide powders;
and mixtures thereof.

The propellants can be chosen from compressed gases or liquefied gases.

As examples of compressed gases, air, nitrogen, carbon dioxide (or carbon dioxide), and their mixtures can be mentioned.

As an example of liquefied gases, dimethyl ether, chlorinated and/or fluorinated hydrocarbons, such as trichlorofluoromethane, dichlorodifluoromethane, chlorodifluoromethane, 1,1,1,2-tetrafluoroethane, chloropentafluoroethane, 1-chloro-1,1-difluoroethane, 1,1-difluoroethane; volatile hydrocarbons, such as especially C3-C5 alkanes, like propane, isopropane, n-butane, isobutane, pentane, alone or in a mixture.

Preferably, hydrocarbons are chosen from propane, isopropane, n-butane, isobutane, alone or in a mixture can be mentioned.

As illustrated in the examples below, the applicant has discovered, surprisingly, that a composition according to the invention comprising at least indole-3-lactic acid and/or a salt thereof; indole-3-carboxaldehyde and/or a salt thereof; and indole-3-acetic acid and/or a salt thereof, makes it possible to overexpress genes for activating the AHR pathway (particularly the genes CYP1A1 and/or OVOL1) and to stimulate differentiation (FLG—Filaggrin and/or DSG1—Desmoglein), and thereby to prevent a reduction in and/or reinforce the barrier function of the skin. This discovery forms the basis of the present invention (see for example Hoober J K, Eggink L L. Int J Mol Sci. 2022 Jan. 27; 23(3):1455). Indeed, filaggrin (FLG) is a major structural protein involved in the surface barrier of the skin. Mutations in the gene encoding filaggrin are the most significant risk factors for skin diseases. Approximately 50% of patients suffering from atopic dermatitis carry mutations which are the source of the loss of function in filaggrin. Esparza-Gordillo et al. (Curr Opin Allergy Clin Immunol. 2010 October; 10(5):418-26) indicated that 10 to 20% of people in industrialized countries suffer from atopic dermatitis, with a strong predisposition in children when the mother carries an FLG mutation. The mutation in the FLG gene is also associated with ichthyosis vulgaris, which is a common skin disease characterized by dry and squamous skin with a prevalence of at least one case in 250 people.

Moreover, DSG1 encodes desmoglein 1, a major constituent of desmosomes, which connect the cell surface to the keratin cytoskeleton and have a crucial role in maintaining the integrity of the epidermis and of the barrier function. The mutations which cause SAM syndrome (severe dermatitis, multiple allergies and metabolic wasting syndrome), a psoriasiform dermatitis, are reflected in an absence of membrane expression of DSG1, leading to a loss of cell-cell adhesion (Oh, J. et al. Nature 514, 59-64 (2014)). A lack of desmoglein 1 leads to severe dermatitis, multiple allergies and a loss of metabolism (Samuelov L et al. Nat Genet. 2013 October; 45(10):1244-1248; Lisa M Godsel et al. J Clin Invest. 2022 Feb. 1; 132(3):e144363.).

These two proteins are therefore key to the barrier role played by the skin.

A composition according to the invention may further comprise tryptamine and/or of a salt thereof in particular at a content of at least 0.00001% by weight relative to the total weight of the composition, and more particularly at least 0.0001% by weight relative to the total weight of the composition.

A composition according to the invention may additionally be characterized in that it comprises:
a content of indole-3-lactic acid and/or a salt thereof of at least 0.00001% by weight relative to the total weight of the composition, in particular of at least 0.0001% by weight relative to the total weight of the composition; and
a content of indole-3-carboxaldehyde and/or a salt thereof of at least 0.00001% by weight relative to the total weight of the composition, in particular of at least 0.0001% by weight relative to the total weight of the composition; and
a content of indole-3-acetic acid and/or a salt thereof of at least 0.000002% by weight relative to the total weight of the composition, in particular of at least 0.00001% by weight relative to the total weight of the composition.

A composition according to the invention may be such that the sum of the contents of indole-3-lactic acid, of indole-3-carboxaldehyde and of indole-3-acetic acid, and/or one of their salts, represents at least 0.0001% by weight relative to the total weight of the composition.

A composition according to the invention may be such that the sum of the contents of indole-3-lactic acid, of indole-3-carboxaldehyde and of indole-3-acetic acid, and/or one of their salts, is between 0.0001% and 10% by weight, in particular between 0.0001% and 1%, more particularly between 0.0001% and 0.1%, even more particularly between 0.0001% to 0.01% by weight relative to the total weight of the composition, for instance 0.00011% by weight relative to the total weight of the composition.

A composition according to the invention may additionally comprise:
a content of indole-3-lactic acid and/or a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, particularly 0.0001% by weight relative to the total weight of the composition;
a content of indole-3-carboxaldehyde and/or a salt thereof ranging from 0.00001% to 10% by weight relative to the total weight of the composition, in particular ranging from 0.0001% to 1%, more particularly ranging from 0.0001% to 0.1%, even more particularly ranging from 0.0001% to 0.01% by weight relative to the total weight of the composition, particularly 0.0001% by weight relative to the total weight of the composition; and
a content of indole-3-acetic acid and/or a salt thereof ranging from 0.000002% to 10% by weight, in particular ranging from 0.00001% to 1% by weight, more particularly ranging from 0.00001% to 0.1% by weight, even more particularly ranging from 0.00001% to 0.01% by weight relative to the total weight of the composition, particularly 0.00001% by weight relative to the total weight of the composition.

A composition according to the invention may comprise a content of tryptamine and/or of a salt thereof of at least 0.00001% by weight relative to the total weight of the composition, and more particularly at least 0.0001% by weight relative to the total weight of the composition.

A composition according to the invention may in particular be such that the [indole-3-lactic acid and/or a salt thereof/indole-3-carboxaldehyde and/or a salt thereof] weight ratio is between 0.5 and 50, in particular between 1 and 10, particularly 1.

A composition according to the invention may in particular be such that the [indole-3-carboxaldehyde and/or a salt thereof/indole-3-acetic acid and/or a salt thereof] weight ratio is between 0.5 and 150, in particular between 1 and 100, more particularly between 1 and 10, particularly 1 or 10.

A composition according to the invention may in particular be such that the [indole-3-lactic acid and/or a salt thereof/indole-3-acetic acid and/or a salt thereof] weight ratio is between 0.5 and 50, in particular between 1 and 10, particularly 1.

A composition according to the invention may in particular be such that the [indole-3-lactic acid and/or a salt thereof/indole-3-carboxaldehyde and/or a salt thereof/indole-3-acetic acid and/or a salt thereof] weight ratio is between 1:1:1 and 10:10:1.

A composition according to the invention may in particular be such that the [indole-3-lactic acid and/or a salt thereof/indole-3-carboxaldehyde and/or a salt thereof/indole-3-acetic acid and/or a salt thereof/tryptamine and/or a salt thereof] weight ratio is between 1:1:1:1 and 10:10:1:10, preferably between 1:1:1:1 and 10:10:1:1.

In particular, a composition according to the invention may comprise a culture supernatant or a fraction of culture supernatant of at least one bacterial strain of the species Staphylococcus epidermidis selected from the group constituted by the bacteria deposited at the CNCM under the numbers I-5689 (deposited by L'Oréal at the CNCM on 3 Jun. 2021), I-5904 (deposited by L'Oréal at the CNCM on 21 Sep. 2022), I-5691 (deposited by L'Oréal at the CNCM on 3 Jun. 2021), I-5693 (deposited by L'Oréal at the CNCM on 3 Jun. 2021), I-5694 (deposited by L'Oréal at the CNCM on 7 Jun. 2021) and I-5695 (deposited by L'Oréal at the CNCM on 7 Jun. 2021).

A composition according to the invention may particularly be suitable for topical administration.

The invention also relates to the cosmetic use, particularly topical cosmetic use, of a composition according to the invention for preventing a reduction in and/or reinforcing the skin barrier function in an individual. For the purposes of the present invention, an individual is preferably a human being.

Moreover, the invention relates to the cosmetic use, particularly topical cosmetic use, of a composition according to the invention for improving skin moisturization.

The invention relates to the cosmetic use, particularly topical cosmetic use, of a composition according to the invention for improving the quality of the skin, in particular of dry skin.

Dry skin appears rough to the touch and appears to be covered with squamae and manifests essentially in a feeling of tautness and/or tension. Indeed, dry skin is generally accompanied by desquamation.

In physiological terms, dry skin is often particularly associated with a decrease in the degree of skin moisturization and with an adverse effect on the barrier function, measured by the insensible water loss. In sensory terms, it is particularly characterized by feelings of skin tautness and/or tension.

Dry skin, also referred to as “xerosis”, may appear at any age, and may be unrelated to a pathological condition.

The invention also relates to the cosmetic use, particularly topical cosmetic use, of a composition according to the invention for preventing and/or treating cosmetic manifestations associated with dry skin, in particular selected from tautness, itching, feelings of discomfort, a dull complexion, skin roughness and a marked microrelief of the skin.

Moreover, the invention relates to the cosmetic use, particularly topical cosmetic use, of a composition according to the invention for preventing and/or treating pruritus, in particular pruritus of skin affected by a dermatological disorder such as atopic dermatitis.

The invention also relates to the cosmetic use, particularly topical cosmetic use, of a composition according to the invention for preventing and/or treating atopic dermatitis.

In the implementations of a composition according to the invention, the composition may in particular be suitable for topical administration.

Finally, the invention relates to a non-therapeutic cosmetic process for caring for keratin materials, in particular the skin, particularly dry skin, particularly dry skin, comprising the topical application to these keratin materials of a composition according to the invention.

Other characteristics, aspects and advantages of the invention will emerge on reading the detailed description that follows.

### BRIEF DESCRIPTION OF THE DRAWINGS

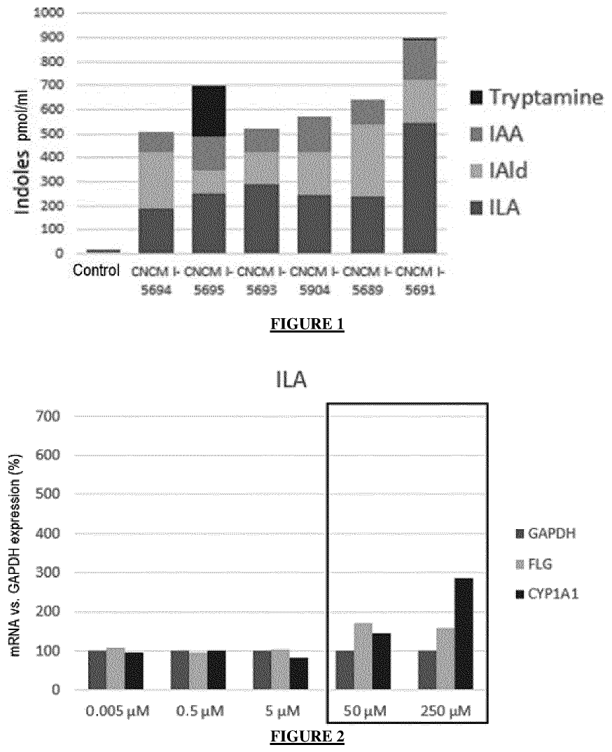

FIG. 1 shows the quantification of the total indole concentrations in the culture supernatants of 6 of the strains tested, namely the reference strains CNCM I-5689, I-5904, I-5691, I-5693, I-5694 and I-5695—i.e. the sums of the concentrations of ILA (indole-3-lactic acid), IAld (indole-3-carboxaldehyde), IAA (indole-3-acetic acid) and tryptamine.
X-axis: from left to right: control, CNCM I-5694, CNCM I-5695, CNCM I-5693, CNCM I-5904, CNCM I-5689 and CNCM I-5691.
Y-axis: total indoles in pmol/ml.

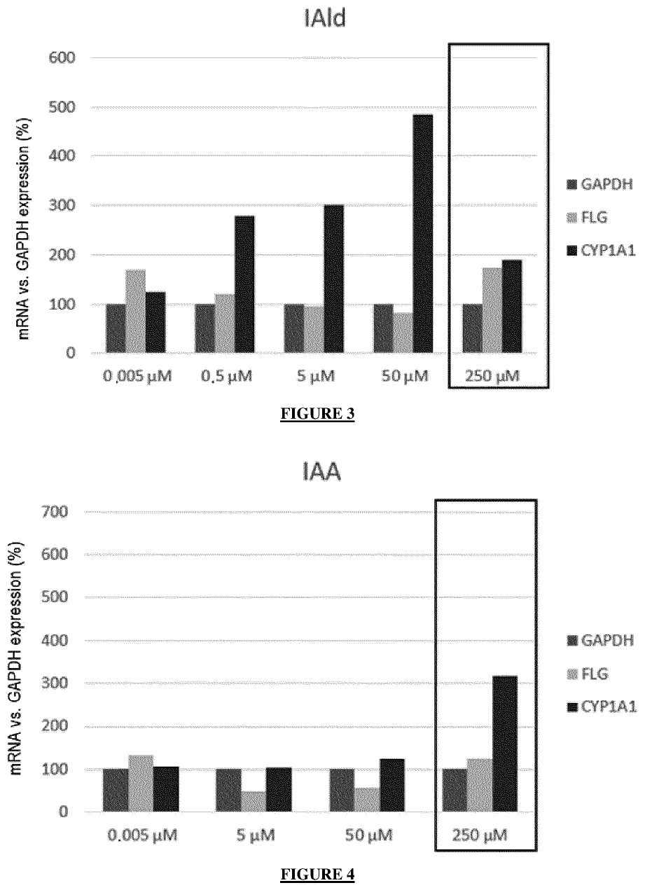

FIG. 2 shows the results of expression of markers of the AHR pathway (CYP1A1) and of the barrier function (FLG) in keratinocytes stimulated by ILA at different concentrations. GAPDH is a reference gene which is a constituent of NHEKs.
X-axis: from left to right: 0.005 μM of ILA; 0.5 μM of ILA; 5 μM of ILA; 50 μM of ILA and 250 μM of ILA. For each concentration, the bars from left to right show GAPDH, FLG then CYP1A1.
Y-axis: percentage (%) expression of GAPDH, FLG and CYP1A1 mRNA compared to GAPDH.

FIG. 3 shows the results of expression of markers of the AHR pathway (CYP1A1) and of the barrier function (FLG) in keratinocytes stimulated by IAld at different concentrations. GAPDH is a reference gene which is a constituent of NHEKs.
X-axis: from left to right: 0.005 μM of IAld; 0.5 μM of IAld; 5 μM of IAld; 50 μM of IAld and 250 μM of IAld. For each concentration, the bars from left to right show GAPDH, FLG then CYP1A1.
Y-axis: percentage (%) expression of GAPDH, FLG and CYP1A1 mRNA compared to GAPDH.

FIG. 4 shows the results of expression of markers of the AHR pathway (CYP1A1) and of the barrier function (FLG) in keratinocytes stimulated by IAA at different concentrations. GAPDH is a reference gene which is a constituent of NHEKs.
X-axis: from left to right: 0.005 μM of IAA; 5 μM of IAA; 50 μM of IAA and 250 μM of IAA. For each concentration, the bars from left to right show GAPDH, FLG then CYP1A1.
Y-axis: percentage (%) expression of GAPDH, FLG and CYP1A1 mRNA compared to GAPDH.

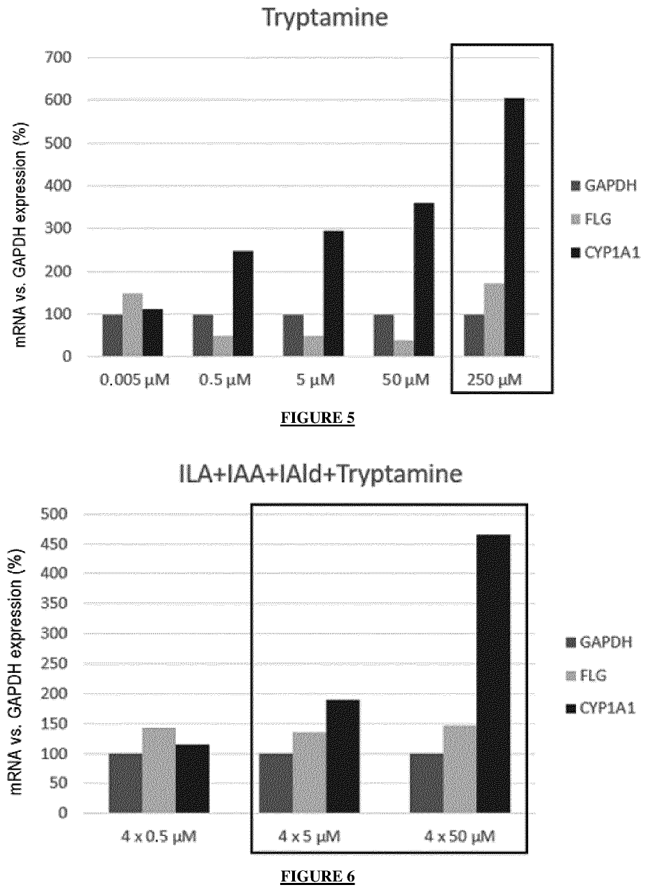

FIG. 5 shows the results of expression of markers of the AHR pathway (CYP1A1) and of the barrier function (FLG) in keratinocytes stimulated by tryptamine at different concentrations. GAPDH is a reference gene which is a constituent of NHEKs.
X-axis: from left to right: 0.005 μM of tryptamine; 0.5 μM of tryptamine; 5 μM of tryptamine; 50 μM of tryptamine and 250 μM of tryptamine. For each concentration, the bars from left to right show GAPDH, FLG then CYP1A1.
Y-axis: percentage (%) expression of GAPDH, FLG and CYP1A1 mRNA compared to GAPDH.

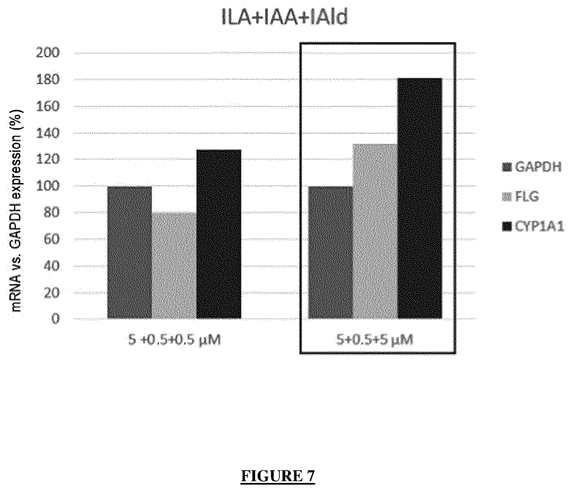

FIG. 6 shows the results of expression of markers of the AHR pathway (CYP1A1) and of the barrier function (FLG) in keratinocytes stimulated by 1:1:1:1 mixtures of ILA, IAld, IAA and tryptamine, at doses of 0.5 (4×0.5 μM), 5 (4×5 μM) or 50 μM (4×50 μM) per compound. GAPDH is a reference gene which is a constituent of NHEKs.
X-axis: from left to right: 4×0.5 μM mixture; 4×5 μM mixture; 4×50 μM mixture. For each concentration, the bars from left to right show GAPDH, FLG then CYP1A1.
Y-axis: percentage (%) expression of GAPDH, FLG and CYP1A1 mRNA compared to GAPDH.

FIG. 7 shows the results of expression of markers of the AHR pathway (CYP1A1) and of the barrier function (FLG) in keratinocytes stimulated by two compositions comprising, respectively, (i) 5 μM of ILA, 0.5 μM of IAld and 0.5 μM of IAA (5+0.5+0.5 μM) or (ii) 5 μM of ILA, 0.5 μM of IAld and 5 μM of IAA (5+0.5+5 μM). GAPDH is a reference gene which is a constituent of NHEKs.
X-axis: from left to right: 5+0.5+0.5 μM mixture; 5+0.5+5 μM mixture. For each concentration, the bars from left to right show GAPDH, FLG then CYP1A1.
Y-axis: percentage (%) expression of GAPDH, FLG and CYP1A1 mRNA compared to GAPDH.

### DETAILED DESCRIPTION

### Composition According to the Invention

A composition according to the invention is preferably cosmetic.

The composition according to the invention is preferentially suitable for topical application to keratin materials, in particular to the skin, and thus comprises a physiologically acceptable medium, i.e. a medium that is compatible with the skin.

The term “cosmetic” means a composition that is compatible with keratin materials, in particular the skin, mucous membranes and skin appendages. The composition according to the invention is non-therapeutic.

The term “keratin materials” is intended to denote in particular the skin, mucous membranes, fibres, eyelashes and skin appendages.

The term “skin” is intended to mean all of the skin of the body, and preferably the skin of the face, scalp, neckline, neck, arms and forearms, or more preferably still the skin of the face, the skin of the face (in particular of the forehead, nose, cheeks and chin), of the neckline and of the neck.

For the purposes of the present invention, the term “physiologically acceptable” is intended to mean a medium which has a pleasant colour, odour and feel and which does not cause any unacceptable discomfort, i.e. stinging or tautness, liable to discourage the user from applying this composition.

As used herein, the terms “treat” and “treatment” mean the alleviation of the symptoms associated with a specific disorder or condition and/or the elimination of said symptoms and also the complete disappearance of the disorder or condition in question.

In the context of the present invention, the terms “prevent” and “prevention” denote the reduction, to smaller degree, of the risk or probability of occurrence of a given phenomenon.

The present invention thus makes it possible to confer beneficial properties on the skin, particularly in a durable manner, in particular: effective barrier function; moisturizing effect; elasticity and smooth texture of the skin; surface morphology, with low roughness, good tissue cohesion and an improvement in the visual appearance of the skin.

In physiological terms, dry skin is often associated with a decrease in the degree of skin moisturization and with an adverse effect on the barrier function, measured by the insensible water loss. In sensory terms, it is particularly characterized by a feeling of tautness, itching, feelings of discomfort and/or skin tension. For obvious reasons, these manifestations cause discomfort.

A composition according to the invention thus proves particularly effective for treating states of skin dryness, for treating dry skin, for treating itching and/or tautness associated with dry skin, for physiologically restoring a suitable state of moisturization of the stratum corneum, for treating hyposeborrheic dry skin, for improving the comfort of dry skin, or else for combating the dull and/or lifeless appearance of the skin as a consequence of its drying-out.

As indicated previously, a composition according to the invention comprises (i) indole-3-lactic acid (ILA) and/or a salt thereof; (ii) indole-3-carboxaldehyde (IAld) and/or a salt thereof; and (iii) indole-3-acetic acid (IAA) and/or a salt thereof.

According to the invention, a “salt” of an indole means a salt formed by an inorganic or organic acid or else an inorganic or organic base.

As examples of acid salts, mention may be made of the sulfate, citrate, acetate, oxalate, chloride, bromide, iodide, nitrate, bisulfate, phosphate, isonicotinate, lactate, salicylate, tartrate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, aspartate and glutamate salts.

As examples of base salts, mention may be made of hydroxides of alkali metals such as sodium, potassium and lithium; hydroxides of alkaline earth metals such as calcium and magnesium; hydroxides of other metals such as aluminium and zinc, aqueous ammonia and organic amines such as unsubstituted or hydroxy-substituted mono-, di- or trialkylamines; dicyclohexylamines; tributylamines, pyridine, N-methyl-N-ethylamine; diethylamine; triethylamine; mono-, bis- or tris(2-hydroxyalkylamines) such as mono-, bis- or tris(2-hydroxyethyl)amine, 2-hydroxy-tert-butylamine or tris(hydroxymethyl)methylamine, N,N-di-alkyl-N-(hydroxyalkyl)-amines, such as N,N-dimethyl-N-(2-hydroxyethyl)amine; N-methyl-D-glucamine; and amino acids such as arginine and lysine.

An indole salt according to the invention is preferably a base salt and in particular a sodium or potassium salt.

According to one variant, in the composition according to the invention, the indole-3-acetic acid is in the form of a base salt, preferably in the form of an inorganic base salt, more preferentially in the form of a salt of alkali metal such as sodium, potassium and lithium, and even more preferentially in the form of a sodium salt.

According to one variant, in the composition according to the invention, the indole-3-carboxaldehyde is present in non-salified form.

According to one variant, in the composition according to the invention, the indole-3-lactic acid is present in non-salified form or in the form of a base salt, preferably in the form of an inorganic base salt, more preferentially in the form of a salt of alkali metal such as sodium, potassium and lithium, even more preferentially in the form of a sodium salt;
preferably, the indole-3-lactic acid is present in non-salified form.

A composition according to the invention preferably comprises (i) indole-3-lactic acid (ILA) or a salt thereof; (ii) indole-3-carboxaldehyde (IAld) or a salt thereof; and (iii) indole-3-acetic acid (IAA) or a salt thereof.

The indole-3-lactic acid (ILA) of a composition according to the invention is of formula (I) below:

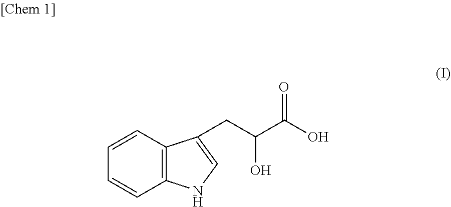 [SMILES: O=C(O)C(O)Cc1c[nH]c2ccccc12]

A composition according to the invention may comprise a content of indole-3-lactic acid and/or of a salt thereof of at least 0.00001% by weight relative to the total weight of the composition, and in particular of at least 0.0001% by weight relative to the total weight of the composition.

A composition according to the invention may comprise a content of indole-3-lactic acid and/or of a salt thereof of at least 0.001% by weight relative to the total weight of the composition, and in particular of at least 0.005% by weight relative to the total weight of the composition.

A composition according to the invention may comprise a content of indole-3-lactic acid and/or of a salt thereof of less than 10% by weight relative to the total weight of the composition, in particular of less than 1% by weight, more particularly of less than 0.1% by weight, even more particularly of less than 0.01% by weight relative to the total weight of the composition.

A composition according to the invention may comprise a content of indole-3-lactic acid and/or of a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition.

The indole-3-carboxaldehyde (IAld) of a composition according to the invention is of formula (II) below:

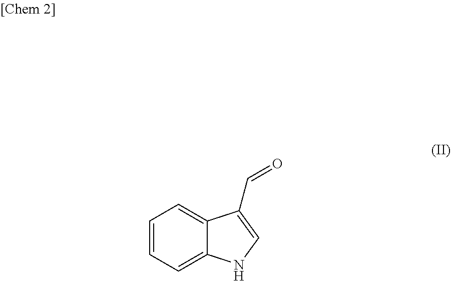 [SMILES: O=Cc1c[nH]c2ccccc12]

A composition according to the invention may comprise a content of indole-3-carboxaldehyde (IAld) and/or of a salt thereof of at least 0.00001% by weight relative to the total weight of the composition, and in particular of at least 0.0001% by weight relative to the total weight of the composition.

A composition according to the invention may comprise a content of indole-3-carboxaldehyde and/or of a salt thereof of at least 0.001% by weight relative to the total weight of the composition, and in particular of at least 0.004% by weight relative to the total weight of the composition.

A composition according to the invention may comprise a content of indole-3-carboxaldehyde and/or of a salt thereof of less than 10% by weight relative to the total weight of the composition, in particular of less than 1% by weight, more particularly of less than 0.1% by weight, even more particularly of less than 0.01% by weight relative to the total weight of the composition.

A composition according to the invention may comprise a content of indole-3-carboxaldehyde and/or a salt thereof ranging from 0.00001% to 10% by weight relative to the total weight of the composition, in particular ranging from 0.0001% to 1% by weight, more particularly ranging from 0.0001% to 0.1%, even more particularly ranging from 0.0001% to 0.01% by weight relative to the total weight of the composition, such as 0.0001% by weight relative to the total weight of the composition.

The indole-3-acetic acid (IAA) of a composition according to the invention is of formula (III) below:

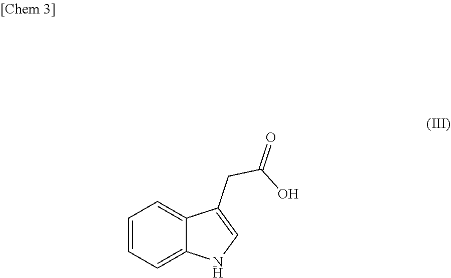 [SMILES: O=C(O)Cc1c[nH]c2ccccc12]

A composition according to the invention may comprise a content of indole-3-acetic acid (IAA) and/or of a salt thereof of at least 0.000002% by weight relative to the total weight of the composition, and in particular of at least 0.00001% by weight relative to the total weight of the composition.

A composition according to the invention may comprise a content of indole-3-acetic acid (IAA) and/or of a salt thereof of at least 0.001% by weight relative to the total weight of the composition, and in particular of at least 0.004% by weight relative to the total weight of the composition.

A composition according to the invention may comprise a content of indole-3-acetic acid and/or of a salt thereof of less than 10% by weight relative to the total weight of the composition, in particular of less than 1% by weight, more particularly of less than 0.1% by weight, even more particularly of less than 0.01% by weight relative to the total weight of the composition.

A composition according to the invention may comprise a content of indole-3-acetic acid and/or of a salt thereof ranging from 0.000002% to 10% by weight, in particular ranging from 0.00001% to 1% by weight, more particularly ranging from 0.00001% to 0.1%, even more particularly ranging from 0.00001% to 0.01% by weight relative to the total weight of the composition, such as 0.00001% by weight relative to the total weight of the composition.

An indole-3-acetic acid (IAA) may in particular be present, entirely or partially, in the form of a base salt, preferably an alkali metal salt, more preferentially a sodium salt, in a composition according to the invention.

In a composition according to the invention, the sum of the contents of indole-3-lactic acid (and/or of a salt thereof), of indole-3-carboxaldehyde (and/or of a salt thereof) and of indole-3-acetic acid (and/or of a salt thereof) may be at least 0.0001% by weight relative to the total weight of the composition, in particular at least 0.00011% by weight relative to the total weight of the composition.

A composition according to the invention may in particular be such that the [indole-3-lactic acid and/or a salt thereof/indole-3-carboxaldehyde and/or a salt thereof] weight ratio is between 0.5 and 50, in particular between 1 and 10, such as 1.

A composition according to the invention may in particular be such that the [indole-3-carboxaldehyde and/or a salt thereof/indole-3-acetic acid and/or a salt thereof] weight ratio is between 0.5 and 150, in particular between 1 and 100, more particularly between 1 and 10, such as 1 or 10.

A composition according to the invention may in particular be such that the [indole-3-lactic acid and/or a salt thereof/indole-3-acetic acid and/or a salt thereof] weight ratio is between 0.5 and 50, in particular between 1 and 10, such as 1.

A composition according to the invention may in particular be such that the [indole-3-lactic acid and/or a salt thereof/indole-3-carboxaldehyde and/or a salt thereof/indole-3-acetic acid and/or a salt thereof] weight ratio is between 1:1:1 and 10:10:1.

In particular, a composition according to the invention may comprise:
a content of indole-3-lactic acid and/or a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition;
a content of indole-3-carboxaldehyde and/or a salt thereof ranging from 0.00001% to 10% by weight relative to the total weight of the composition, in particular ranging from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition; and
a content of indole-3-acetic acid and/or a salt thereof, and in particular a content of sodium salt, ranging from 0.000002% to 10% by weight, in particular ranging from 0.00001% to 1% by weight, more particularly ranging from 0.00001% to 0.1% by weight, even more particularly ranging from 0.00001% to 0.01% by weight relative to the total weight of the composition, such as 0.00001% by weight relative to the total weight of the composition.

A composition according to the invention may further comprise tryptamine and/or a salt thereof.

The tryptamine is of formula (IV) below:

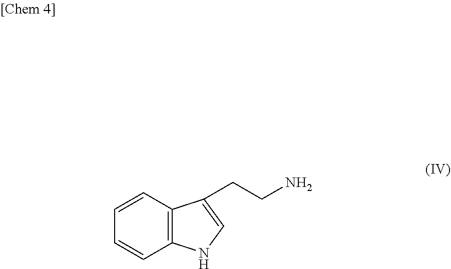 [SMILES: NCCc1c[nH]c2ccccc12]

Tryptamine and/or a salt thereof may be present in a composition according to the invention at a content of at least 0.00001% by weight relative to the total weight of the composition, and in particular of at least 0.0001% by weight relative to the total weight of the composition.

A composition according to the invention may comprise a content of tryptamine and/or of a salt thereof of less than 10% by weight relative to the total weight of the composition, in particular of less than 1% by weight, more particularly of less than 0.1% by weight, even more particularly of less than 0.01% by weight relative to the total weight of the composition.

A composition according to the invention may comprise a content of tryptamine and/or of a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition.

In particular, a composition according to the invention may comprise:
a content of indole-3-lactic acid and/or a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition; and
a content of indole-3-carboxaldehyde and/or a salt thereof ranging from 0.00001% to 10% by weight relative to the total weight of the composition, in particular ranging from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition; and
a content of indole-3-acetic acid and/or a salt thereof, and in particular a content of sodium salt, ranging from 0.000002% to 10% by weight, in particular ranging from 0.00001% to 1% by weight, more particularly ranging from 0.00001% to 0.1% by weight, even more particularly ranging from 0.00001% to 0.01% by weight relative to the total weight of the composition, such as 0.00001% by weight relative to the total weight of the composition; and
a content of tryptamine and/or a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, such as 0.0001% by weight relative to the total weight of the composition.

In a composition according to the invention, the sum of the contents of indole-3-lactic acid (and/or of a salt thereof), of indole-3-carboxaldehyde (and/or of a salt thereof) and of indole-3-acetic acid (and/or of a salt thereof) and of tryptamine (and/or of a salt thereof) may be at least 0.0003% by weight relative to the total weight of the composition.

A composition according to the invention may in particular be such that the [indole-3-lactic acid and/or a salt thereof/indole-3-carboxaldehyde and/or a salt thereof/indole-3-acetic acid and/or a salt thereof/tryptamine and/or a salt thereof] weight ratio is between 1:1:1:1 and 10:10:1:10, preferably between 1:1:1:1 and 10:10:1:1.

A composition according to the invention may in particular comprise a culture supernatant or a fraction of culture supernatant of at least one bacterial strain that produces indole-3-lactic acid and/or a salt thereof, indole-3-carboxaldehyde and/or a salt thereof and indole-3-acetic acid and/or a salt thereof, and also optionally tryptamine and/or a salt thereof, and in particular also tryptamine and/or a salt thereof.

A composition according to the invention may in particular comprise a culture supernatant or a fraction of culture supernatant of at least one bacterial strain of the species Staphylococcus epidermidis that produces indole-3-lactic acid and/or a salt thereof, indole-3-carboxaldehyde and/or a salt thereof and indole-3-acetic acid and/or a salt thereof, and also optionally tryptamine and/or a salt thereof, and in particular also tryptamine.

Thus, a composition according to the invention preferably comprises a culture supernatant or a fraction of culture supernatant of at least one bacterial strain of the species Staphylococcus epidermidis selected from the group constituted by bacteria deposited at the CNCM under the numbers I-5689, I-5904, I-5691, I-5693, I-5694 and I-5695.

A composition according to the invention additionally comprises at least one adjuvant selected from the group constituted by fatty substances, organic solvents selected from lower monoalcohols having from 1 to 5 carbon atoms such as ethanol, isopropanol, tert-butanol; polyols such as propylene glycol, pentylene glycol, caprylyl glycol, hexylene glycol (or 2-methyl-2,4-pentanediol) and polyethylene glycols; polyol ethers such as dipropylene glycol monomethyl ether, ionic or nonionic, hydrophilic or lipophilic, thickeners; softeners; opacifiers; stabilizers; silicones; antifoams; fragrances; preservatives; anionic, cationic, nonionic, zwitterionic or amphoteric surfactants; fillers; polymers; propellants; and mixtures thereof.

Such an adjuvant may represent from 0.0001% to 20%, preferably from 0.01% to 10% and better still from 0.1% to 5% by weight relative to the total weight of the composition.

Of course, those skilled in the art will take care to select this or these adjuvant(s), and/or the amount thereof, such that the advantageous properties of the indole derivatives of the composition according to the invention are not, or are not substantially, adversely affected by the envisaged addition.

According to one embodiment, the composition according to the invention may comprise water.

In particular, a composition according to the invention may comprise from 20% to 90% by weight and preferably from 30% to 60% by weight of water, relative to the total weight of the composition.

The composition according to the invention may further comprise at least one additional cosmetic active agent.

This may in particular be at least one active agent for caring for dry skin, such as glycerol or urea.

In the context of the present invention, the term “additional active agent” means a compound which acts by itself, that is to say which does not require the intervention of an external agent in order to activate it.

The additional active agent which can be used in the compositions of the invention may in particular be selected from desquamating agents, soothing agents, anti-irritant agents, anti-ageing agents, wound-healing agents, antibacterial agents, vitamins, and mixtures thereof in any proportions.

The additional active agent used in the composition according to the invention may represent from 0.0001% to 20%, preferably from 0.01% to 10% and better still from 0.1% to 5% by weight relative to the total weight of the composition.

Needless to say, a person skilled in the art will take care to select this or these optional additional compound(s), and/or the amount thereof, such that the advantageous properties of the composition according to the invention are not, or are not substantially, adversely affected by the envisioned addition.

A composition according to the invention may be in any presentation form normally used in the cosmetics field.

It may particularly be in the form of an aqueous or aqueous-alcoholic solution, which may be gelled, a dispersion of the lotion type, which may be a two-phase dispersion, an oil-in-water or water-in-oil emulsion or a multiple emulsion, an aqueous gel, or else a dispersion of oils in an aqueous phase, particularly using spherules, it being possible for these spherules to be polymeric particles or, better still, lipid vesicles of ionic and/or nonionic type. It may be of more or less fluid liquid consistency.

Preferentially, a composition according to the invention differs from compositions having an essentially detergent purpose with regard to the skin, hair and/or mucous membranes, such as soaps, shampoos and shower gels for washing and/or cleansing.

A composition according to the invention is preferentially suitable for topical administration.

Thus, a composition according to the invention may comprise all the constituents usually employed in the envisaged topical application and administration.

A composition according to the invention may advantageously be in the form of an emulsion, particularly obtained by dispersion of an aqueous phase in a fatty phase (W/O) or of a fatty phase in an aqueous phase (O/W), of liquid or semi-liquid consistency of the milk type, or of soft consistency, or even of multiple emulsion (W/O/W or O/W/O). These compositions are prepared according to the usual known methods.

More particularly, a composition according to the invention may be intended for topical application and may preferably be in the form of an emulsion, preferably an oil-in-water emulsion. Preferably, such an emulsion is not intended to be rinsed off after application.

A composition according to the invention is preferentially intended to be applied to skin.

Preferably, the skin is the skin of the face, scalp, neckline, neck, arms or forearms, or even more preferably the skin of the face (in particular of the forehead, nose, cheeks and chin), neckline and neck.

The pH of said composition is advantageously less than or equal to 8, preferably ranging from 4 to 7, better still ranging from 4.5 to 6.5.

The composition may alternatively be in the form of a face and/or body care or makeup product, and may be packaged, for example, in the form of a cream in a jar or a fluid in a tube or a pump bottle or a dropper bottle.

The composition according to the invention may be manufactured via any known process generally used in the cosmetics field.

The ingredients are mixed, before being formed, in the order and under conditions that may readily be determined by a person skilled in the art.

According to a particular form of the invention, other agents intended to enhance the appearance and/or the texture of the skin may also be added to the composition according to the invention.

### Uses and Processes

According to one of its aspects, the present invention relates to the cosmetic use, particularly topical cosmetic use, of a composition according to the invention for preventing a reduction in and/or reinforcing the skin barrier function in an individual.

According to yet another one of its aspects, the present invention relates to the cosmetic use, particularly topical cosmetic use, of a composition according to the invention for improving skin moisturization.

The present invention also relates to the cosmetic use, particularly topical cosmetic use, of a composition according to the invention for improving the quality of the skin, in particular of dry skin.

The present invention also relates to the cosmetic use, particularly topical cosmetic use, of a composition according to the invention for preventing and/or treating cosmetic manifestations associated with dry skin, in particular selected from tautness, itching, feelings of discomfort, a dull complexion, skin roughness and a marked microrelief of the skin.

Moreover, one subject of the present invention relates to the cosmetic use, particularly topical cosmetic use, of a composition according to the invention for preventing and/or treating pruritus, in particular pruritus of skin affected by a dermatological disorder such as atopic dermatitis.

It also relates to the cosmetic use, particularly topical cosmetic use, of such a composition for preventing and/or treating atopic dermatitis.

According to yet another one of its aspects, the present invention relates to a non-therapeutic cosmetic process for caring for keratin materials, in particular the skin, particularly dry skin, comprising the topical application to these keratin materials of a composition according to the invention.

The cosmetic uses and processes considered according to the invention are non-therapeutic.

The cosmetic uses and processes of the invention are preferentially implemented by topically administering a composition according to the invention.

Topical administration consists of the external application to the skin of cosmetic compositions according to the usual techniques for the use of these compositions.

By way of illustration, the cosmetic use or process according to the invention may be implemented by topical, for example daily, application of at least one composition according to the invention, which may be formulated, for example, in the form of a cream, gel, serum, lotion, emulsion, or makeup-removing milk, preferably in the form of an emulsion.

The application can be repeated, for example, once to twice daily for one or more days and generally over an extended period of at least 4 weeks, or even 4 to 15 weeks, with, where appropriate, one or more periods of stoppage.

According to one embodiment, the application is daily (once a day) and generally over an extended period of at least 4 weeks, or even 4 to 15 weeks, with, where appropriate, one or more periods of stoppage.

According to one embodiment, the cosmetic treatment process according to the invention may comprise a single application.

Throughout the description, including the claims, the terms “between . . . and . . . ”, and “ranging from . . . to . . . ” should be understood as meaning that the limits are included, unless otherwise specified.

The examples that follow illustrate the present invention without limiting the scope thereof.

In the examples, unless otherwise specified, the temperature is room temperature (20° C.) and is expressed in degrees Celsius, and the pressure is atmospheric pressure.

### EXAMPLES

### Materials and Methods

### Origin of the Strains, CNCM Names and Culture Methods

The Staphylococcus epidermidis strains selected, numbering 8, were collected from normal healthy subjects. The samples were obtained using the swabbing method, as described previously, for example, in Leung, M. H. Y. et al. Changes of the human skin microbiota upon chronic exposure to polycyclic aromatic hydrocarbon pollutants. Microbiome 8, 100 (2020). The bacteria collected were isolated on tryptone soya agar (TSA) and preserved in the form of frozen stock.

These six strains are as follows: the Staphylococcus epidermidis (S. epidermidis) strain deposited by L'Oréal at the CNCM on 3 Jun. 2021 under accession number CNCM I-5689 (I-5689), the Staphylococcus epidermidis (S. epidermidis) strain deposited by L'Oréal at the CNCM on 21 Sep. 2022 under accession number CNCM I-5904 (I-5904), the Staphylococcus epidermidis (S. epidermidis) strain deposited by L'Oréal at the CNCM on 3 Jun. 2021 under accession number CNCM I-5691 (I-5691), the Staphylococcus epidermidis (S. epidermidis) strain deposited by L'Oréal at the CNCM on 3 Jun. 2021 under accession number CNCM I-5693 (I-5693), the Staphylococcus epidermidis (S. epidermidis) strain deposited by L'Oréal at the CNCM on 7 Jun. 2021 under accession number CNCM I-5694 (I-5694) and the Staphylococcus epidermidis (S. epidermidis) strain deposited by L'Oréal at the CNCM on 7 Jun. 2021 under accession number CNCM I-5695 (I-5695).

The bacterial strains were cultured either in TSB (tryptic soy broth) or in KSFM (keratinocyte serum-free medium) using conventional microbiology methods.

### Culture and Treatments of Keratinocytes

Normal neonatal human primary epidermal keratinocytes (NHEKs) (CellnTec) were cultured in a medium supplemented with CnT-57 (CellnTec) containing a bovine pituitary extract (BPE) at 37° C. and 5% CO₂. The NHEKs were cultured to approximately 90% confluence in keratinocyte-SFM medium (Gibco, Life Tech. Corp., Grand Island, USA) supplemented with 0.035 μg/μl EGF and 12.4 mg/ml BPE (Gibco) 24 hours before the treatments. For the treatments with bacterial supernatant, the NHEKs were treated with sterilized bacterial supernatant at 50% (v/v) as final concentration in the KSFM medium and incubated for 14 hours, then the NHEKs were subsequently harvested for RNA extraction. At the end of the treatment, the media were collected for the quantification of IL-8 (IL-8 DuoSet ELISA, R&D systems) and the cytotoxicity assay (CyQuant™ LDH cytotoxicity assay, Thermo Fisher), and the cells were harvested for the real-time quantitative PCR assay.

### Real-Time Quantitative PCR

The total RNA was extracted from the keratinocytes using the RNeasy Micro kit (Qiagen, Hilden, Germany) following the manufacturer's protocol, with an additional treatment with DNase (Rnase-Free Dnase Set, Qiagen). The RNA concentration was determined using a Nanodrop™ (Nanodrop 1000, Thermo Scientific). iScript cDNA Synthesis (Biorad) was used to synthesize the cDNA. The mRNA levels were measured using the StepOnePlus™ Real-Time PCR system (Applied Biosystems™) and SYBR Green Master Mix (Biorad). The data from the quantitative PCR (qPCR) were analyzed using the 2-AACt quantification method, with GAPDH (glyceraldehyde-3-phosphate dehydrogenase) for the eukaryotic cells and GyrB (DNA gyrase subunit B) for the staphylococcus as endogenous control. Primers for FLG, IVL, KLK7, DSG1, CDSN, Ki67, DEFB4, STAT6, OVOL1, OVOL2 were provided by Biorad, the other primer sequences are presented below:

Target gene CYP1A1: sense primer (5′-3′) of
sequence SEQ ID NO: 1:
(SEQ ID NO: 1)
CAGCTCAGCTCAGTACCTCA
and antisense primer (3′-5′) of sequence
SEQ ID NO: 2:
(SEQ ID NO: 2)
CTTGAGGCCCTGATTACCCA.
Target gene GyrB Se: sense primer (5′-3′) of
sequence SEQ ID NO: 3:
(SEQ ID NO: 3)
GTTGTAATTGAGAAAGACAATTG
and antisense primer (3′-5′) of sequence
SEQ ID NO: 4:
(SEQ ID NO: 4)
TACAGTTAAGATAACTTCGACAG.

### Quantification of AHR Metabolites/Ligands

Preparation: 5-Hydroxyindole-acetic acid-d5, serotonin-d4, indole-3-acetic acid-d4, kynurenic acid-d5, melatonin-d4, piconilic acid-d3, tryptamine-d4 and xanthurenic acid-d4 were purchased from Santa Cruz Biotechnology. 3-Hydroxyantrhanilic acid-d4, 3-hydroxykynurenin-13C2-15N, 5-hydroxytryptophan-d4, indole-3-acetamide-d5, kynurenin-d4 and tryptophan-d5 were purchased from Toronto Research Chemicals.

The stock solutions of the labelled analytes were prepared in water with 0.1% formic acid and the end concentrations were chosen to correspond to the estimated concentrations of endogenous metabolites.

Extraction of metabolites: The metabolites were extracted from 50 μl of medium, collected as described above. After adding the preparations indicated above (100 μl) and 300 μl of MeOH, the samples were mixed for 15 s and homogenized at −20° C., for 30 min. After centrifugation for 10 min at 5000 rpm and −4° C., 350 μl of supernatant were collected and concentrated under a stream of N2. The residues were reconstituted in 100 μl of MeOH/H2O (1:9) and transferred to a 96-well plate for LC-HRMS.

Instrumentation: The analyses were carried out as described previously in Lefevre, A. et al., Talanta 195, 593-598 (2019). In brief, 2 μl were injected into the LC-MS (XEVO-TQ-XS, Waters®). A Kinetex C18 xb column (1.7 μm×150 mm×2.1 mm, temperature 55° C.) associated with a gradient of two mobile phases (Phase A: water+0.5% formic acid; Phase B: MeOH+0.5% formic acid) at a flow rate of 0.4 ml/min were used.

For each metabolite, calibration curves were used to determine the concentrations of each metabolite in the samples.

### Statistical Analysis

All experiments were performed with at least three biological replicates. The data is presented in the form of mean±SEM. GraphPad Prism (version 7.0; GraphPad Software, La Jolla, California). The data was analyzed using the Kruskal-Wallis test followed by Dunn's test. The results are considered to be statistically significant when p<0.05.

### Example 1: Selection of 6 Staphylococcus epidermidis Strains on the Basis of their Secretion of Indole in the Culture Supernatants

This selection of Staphylococcus epidermidis strains is based on their secretion of indoles in the culture supernatants when they are cultured alone, and on the ability of the supernatant to activate the AHR pathway and the genes of the skin barrier function.

Multiple unique strains are cultured in TSB for 16 hours, then the supernatant is collected, in order firstly to quantify the indole metabolites and then to treat the keratinocytes and carry out Rt-qPCR analysis of the targeted genes, following the protocols described in detail above.

### Results

Quantification of the concentrations of indoles in the culture supernatants indicated a concentration greater than 443 pmol/ml of total indoles—i.e. the sum of the concentration of ILA (indole-3-lactic acid), IAld (indole-3-carboxaldehyde), IAA (indole-3-acetic acid) and tryptamine—for 6 of the strains tested, namely the strains previously indicated having the references I-5689, I-5904, I-5691, I-5693, I-5694 and I-5695 (see Table 1 below and FIG. 1).

TABLE 1

| | Indole-3- | Indole-3- | Indole-3- | | Sum of |
|---|---|---|---|---|---|
| | lactic acid | carboxaldehyde | acetic acid | Tryptamine | indoles |
| Conditions | (pmol/ml) | (pmol/ml) | (pmol/ml) | (pmol/ml) | (pmol/ml) |
| Culture medium | <2.75 | 9.25 | <1.6 | 1.91 | 11.156 |
| NHEKs (KSFM) | | | | | |
| Culture medium | <2.75 | 9.43 | <1.6 | 1.98 | 11.414 |
| NHEKs (KSFM + | | | | | |
| TSB (50/50 v/v)) | | | | | |
| CNCM I-5694 | 190.46 | 231.61 | 82.75 | <0.145 | 504.82 |
| CNCM I-5695 | 249.14 | 95.95 | 143.74 | 207.53 | 696.35 |
| CNCM I-5693 | 291.95 | 133.91 | 96.45 | <0.145 | 522.306 |
| CNCM I-5904 | 241.91 | 184.47 | 145.61 | <0.145 | 571.99 |
| CNCM I-5689 | 237.28 | 300.25 | 101.96 | <0.145 | 639.496 |
| CNCM I-5691 | 548.05 | 175.11 | 165.88 | 5.70 | 894.748 |

Not only do the supernatants from each of these strains lead to overexpression of CYP1A1 (>2.7 times), they also lead to overexpression of OVOL1 compared to untreated cells.

TABLE 2

| | Average level of mRNA expression |
|---|---|
| | of the gene CYP1A1 vs. GAPDH |
| | (reference gene which is a |
| Conditions | constituent of NHEKs) |
| Control (untreated NHEKs) | 1 |
| CNCM I-5694 | 5.418 |
| CNCM I-5695 | 4.678 |
| CNCM I-5693 | 6.594 |
| CNCM I-5689 | 8.987 |
| CNCM I-5904 | 36.81 |
| CNCM I-5691 | 6.308 |

TABLE 3

| | Average level of mRNA expression |
|---|---|
| | of the gene OVOL1 vs. GAPDH |
| | (reference gene which is a |
| Conditions | constituent of NHEKs) |
| Control (untreated NHEKs) | 1 |
| CNCM I-5694 | 1.498 |
| CNCM I-5695 | 1.65 |
| CNCM I-5693 | 2.258 |
| CNCM I-5689 | 1.46 |
| CNCM I-5904 | 4.974 |
| CNCM I-5691 | 1.52 |

### Example 2: Particular Compositions of Indoles that Mimic the Presence of Bacteria and Maintain the Skin Barrier Function

This example describes compositions of indoles that activate the AHR pathway and maintain the skin barrier function.

An effective mixture of indoles is a composition which leads to overexpression of the genes for AHR pathway activation (specifically the genes CYP1A1 and OVOL1, described by Furue et al. Int. J. Mol. Sci. 20, (2019)) and to stimulation of differentiation (FLG—filaggrin).

No cytotoxicity and no inflammation (IL-8) were measured with any of the indoles or indole mixtures tested.

A. Different concentrations were firstly tested according to the protocol indicated previously, for the four indoles alone, i.e: 0.005 μM; 0.5 μM; 5 μM; 50 μM; or 250 μM for ILA, IAld, and tryptamine; 0.005 μM; 5 μM; 50 μM; or 250 μM for IAA.

The results of expression of markers of the AHR pathway (CYP1A1) and of the barrier function (FLG) in the keratinocytes stimulated by the indoles alone are represented in FIG. 2 (ILA), FIG. 3 (IAld), FIG. 4 (IAA) and FIG. 5 (tryptamine).

Thus, apart from ILA for which the minimum active concentration is approximately 50 μM, the three other indoles, when used alone, each have a minimum active concentration of 250 μM.

B. Different mixtures of these indoles were also additionally tested according to the protocol indicated previously.

a. Firstly, a 1:1:1:1 mixture of ILA, IAld, IAA and tryptamine at doses of 0.5, 5 or 50 μM per compound (i.e. 3 experiments at total indole concentrations of 2, 20 and 200 PM).

The results obtained are shown in FIG. 6.

Unexpectedly, a minimum active concentration of this mixture is observed from 20 μM (5 μM of ILA+5 μM of IAld+5 μM of IAA+5 μM of tryptamine).

This concentration is the concentration required to obtain activity which was only obtained with the individual compounds starting at 50 μM for ILA and 250 μM for the 3 other indoles.

Therefore, surprisingly, this composition is observed to be superior to the individual compounds.

b. Different mixtures comprising ILA, IAld and IAA were also tested according to the protocol indicated previously.

Thus, two compositions comprising, respectively, (i) 5 μM of ILA, 0.5 μM of IAld and 0.5 μM of IAA, or (ii) 5 μM of ILA, 0.5 μM of IAld and 5 μM of IAA were tested.

The results obtained are shown in FIG. 7.

Unexpectedly, a minimum active concentration of this mixture is observed from 10.5 μM (5 μM of ILA+0.5 μM of IAld+5 μM of IAA in sodium salt form).

This concentration is the concentration required to obtain activity which was only obtained with the individual compounds starting at 50 μM for ILA and 250 μM for the 3 other indoles. Moreover, this activity is obtained despite the absence of tryptamine.

Therefore, surprisingly, this composition is observed to be superior to the individual compounds.

1. A composition for caring for keratin materials, comprising, in a physiologically acceptable medium:
at least indole-3-lactic acid and/or a salt thereof; indole-3-carboxaldehyde and/or a salt thereof; and indole-3-acetic acid and/or a salt thereof; and
at least one adjuvant selected from the group constituted by fatty substances; organic solvents selected from lower monoalcohols having from 1 to 5 carbon atoms such as ethanol, isopropanol, tert-butanol; polyols such as propylene glycol, pentylene glycol, caprylyl glycol, hexylene glycol (or 2-methyl-2,4-pentanediol) and polyethylene glycols; polyol ethers such as dipropylene glycol monomethyl ether; ionic or nonionic, hydrophilic or lipophilic, thickeners; softeners; opacifiers; stabilizers; silicones; antifoams; fragrances; preservatives; anionic, cationic, nonionic, zwitterionic or amphoteric surfactants; fillers; polymers; propellants; and mixtures thereof.

2. The composition according to claim 1, further comprising tryptamine and/or a salt thereof.

3. The composition according to claim 1, comprising:
a content of indole-3-lactic acid and/or a salt thereof of at least 0.00001% by weight relative to the total weight of the composition, in particular of at least 0.0001% by weight relative to the total weight of the composition; and
a content of indole-3-carboxaldehyde and/or a salt thereof of at least 0.00001% by weight relative to the total weight of the composition, in particular of at least 0.0001% by weight relative to the total weight of the composition; and
a content of indole-3-acetic acid and/or a salt thereof of at least 0.000002% by weight relative to the total weight of the composition, in particular of at least 0.00001% by weight relative to the total weight of the composition.

4. The composition according to any one of claim 1, wherein the sum of the contents of indole-3-lactic acid, of indole-3-carboxaldehyde and of indole-3-acetic acid, and/or of one of their salts, represents at least 0.0001% by weight relative to the total weight of the composition.

5. The composition according to claim 2, comprising:
a content of indole-3-lactic acid and/or a salt thereof ranging from 0.00001% to 10% by weight relative to the total weight of the composition, in particular from 0.0001% to 1%, more particularly ranging from 0.0001% to 0.1%, even more particularly ranging from 0.0001% to 0.01% by weight relative to the total weight of the composition, particularly 0.0001% by weight relative to the total weight of the composition;
a content of indole-3-carboxaldehyde and/or a salt thereof ranging from 0.00001% to 10% by weight relative to the total weight of the composition, in particular ranging from 0.0001% to 1%, more particularly ranging from 0.0001% to 0.1%, even more particularly ranging from 0.0001% to 0.01% by weight relative to the total weight of the composition, particularly 0.0001% by weight relative to the total weight of the composition; and
a content of indole-3-acetic acid and/or a salt thereof ranging from 0.000002% to 10% by weight, in particular ranging from 0.00001% to 1% by weight, more particularly ranging from 0.00001% to 0.1% by weight, even more particularly ranging from 0.00001% to 0.01% by weight relative to the total weight of the composition, particularly 0.00001% by weight relative to the total weight of the composition.

6. The composition according to claim 2, wherein the content of tryptamine and/or of a salt thereof is at least 0.00001% by weight relative to the total weight of the composition, and more particularly at least 0.0001% by weight relative to the total weight of the composition.

7. The composition according to claim 1, wherein the [indole-3-lactic acid and/or a salt thereof/indole-3-carboxaldehyde and/or a salt thereof] weight ratio is between 0.5 and 50, in particular between 1 and 10, particularly 1.

8. The composition according to claim 1, wherein the [indole-3-carboxaldehyde and/or a salt thereof/indole-3-acetic acid and/or a salt thereof] weight ratio is between 0.5 and 150, in particular between 1 and 100, more particularly between 1 and 10, particularly 1 or 10.

9. The composition according to claim 1, characterized in that the [indole-3-lactic acid and/or a salt thereof/indole-3-acetic acid and/or a salt thereof] weight ratio is between 0.5 and 50, in particular between 1 and 10, particularly 1.

10. The composition according to claim 1, wherein the [indole-3-lactic acid and/or a salt thereof/indole-3-carboxaldehyde and/or a salt thereof/indole-3-acetic acid and/or a salt thereof] weight ratio is between 1:1:1 and 10:10:1.

11. The composition according to claim 1, wherein the [indole-3-lactic acid and/or a salt thereof/indole-3-carboxaldehyde and/or a salt thereof/indole-3-acetic acid and/or a salt thereof/tryptamine and/or a salt thereof] weight ratio is between 1:1:1:1 and 10:10:1:10, preferably between 1:1:1:1 and 10:10:1:1.

12. The composition according to claim 1, the composition comprising a culture supernatant, or a fraction of culture supernatant, of at least one bacterial strain of the species Staphylococcus epidermidis selected from the group constituted by bacteria deposited at the CNCM under the numbers I-5689, I-5904, I-5691, I-5693, I-5694 and I-5695.

13. The composition according to claim 1, the composition being suitable for topical administration.

14. A cosmetic use, in particular topical cosmetic use, of a composition according to claim 1 for preventing a reduction in and/or reinforcing the skin barrier function in an individual.

15. A cosmetic use, in particular topical cosmetic use, of a composition according to claim 1 for improving skin moisturization.

16. A cosmetic, in particular topical cosmetic use, of a composition according to claim 1 for improving the quality of the skin, in particular of dry skin.

17. A non-therapeutic cosmetic use, in particular topical non-therapeutic cosmetic use, of a composition according to claim 1 for preventing and/or treating cosmetic manifestations associated with dry skins, in particular selected from tautness, itching, feelings of discomfort, a dull complexion, skin roughness and a marked microrelief of the skin.

18. A non-therapeutic cosmetic use, particularly topical non-therapeutic cosmetic use, of a composition according to claim 1, for preventing and/or treating pruritus, in particular pruritus of skin affected by a dermatological disorder such as atopic dermatitis.

19. The composition according to claim 1, for use thereof in the prevention and/or treatment of atopic dermatitis.

20. The cosmetic use according to claim 14, or composition for use thereof according to claim 19, wherein the composition is suitable for topical administration.

21. The non-therapeutic cosmetic process for caring for keratin materials, in particular the skin, particularly dry skin, comprising the topical application to these keratin materials of a composition according to claim 1.
